(19) United States
(12) Patent Application Publication
(10) Pub. No.: US 2024/0173212 A1
(43) Pub. Date: May 30, 2024

(54) HIGH TRANSPARENCY AND LONG-RELEASE DENTAL RESIN COMPOSITION CONTAINING CORE-SHELL POROUS STRUCTURED NANOPARTICLES AND METHOD FOR PREPARING THE SAME

(71) Applicants: JNP Nature Co., Ltd., Incheon (KR); MIMETICS.Co., Ltd., Suwon-si (KR)

(72) Inventors: Youngseok JEON, Goyang-si (KR); Hyoungki Park, Suwon-si (KR); Kisu Park, Suwon-si (KR); Geonyeong Lee, Suwon-si (KR)

(73) Assignees: JNP Nature Co., Ltd., Incheon (KR); MIMETICS.Co., Ltd., Suwon-si (KR)

(21) Appl. No.: 18/458,487

(22) Filed: Aug. 30, 2023

(30) Foreign Application Priority Data
Nov. 25, 2022 (KR) 10-2022-0160358

(51) Int. Cl. A61K 6/20; A61K 6/17; A61K 6/60; A61K 6/62; A61K 6/69

(52) U.S. Cl. CPC A61K 6/20; A61K 6/17; A61K 6/60; A61K 6/62; A61K 6/69

(57) ABSTRACT

Disclosed is a highly transparent and long-release dental resin composition containing core-shell porous structured nanoparticles and a method for preparing the same. More specifically, disclosed is a pigment-free and dye-free photocurable dental resin composition containing nanoparticles, which contains a core portion with an oral active ingredient and a porous ceramic shell portion coated on a part or all of a surface of the core portion, and a method for producing the same.

The dental resin composition according to the present disclosure has excellent color compatibility with natural teeth and releases an oral active ingredient contained in a porous structure for a long period of time, and thus, the dental resin composition may be used as an aesthetic/medical multifunctional complex composition.

### BACKGROUND OF INVENTION

### Field of the Invention

The present disclosure relates to a highly transparent and long-release dental resin composition containing core-shell porous structured nanoparticles and a method for preparing the same. More specifically, the present disclosure relates to a pigment-free and dye-free photocurable dental resin composition containing nanoparticles, which contains a core portion with an oral active ingredient and a porous ceramic shell portion coated on a part or all of a surface of the core portion, and a method for producing the same.

The dental resin composition according to the present disclosure has excellent color compatibility with natural teeth and releases an oral active ingredient contained in a porous structure for a long period of time, and thus, the dental resin composition may be used as an aesthetic/medical multifunctional complex composition.

### Related Art

Conventionally, in various fields such as dental materials, recording materials, and building materials, a curable composition containing a polymerizable monomer and an inorganic or organic filler has been used. In particular, in the field of dental filling restoration materials, curable compositions have rapidly spread as a material for restoring teeth damaged by caries, fracture, etc., because the curable compositions can impart a color tone equivalent to natural tooth color and are easy to operate. In recent years, with improved mechanical strength and improved adhesion to teeth, the curable compositions have been used not only for restoration of anterior teeth, but also for molars to which high bite pressure is applied.

A conventional photopolymerizable composition for dental restoration consists of a synthetic monomer, an organic/inorganic filler, a photopolymerization initiator, a polymerization inhibitor, a stabilizer, a colorant such as dye/pigment, and the like. When the colorant such as dye/pigment are included, the transparency is very poor, which leads to problems regarding tooth gloss, color contrast with neighboring teeth, discoloration of a dental restorative material due to deterioration of a colorant, and color change before and after photocuring. In addition, in the case of organic/inorganic fillers, product quality control has not been properly performed due to the amorphous and non-regular size, shape, and constituents. Also, the development of 30 types of dental restorative resins by classifying human tooth colors into A (Reddish-Brownish), B (Reddish-Yellowish), C (Greyish Shades), and D (Reddish-Grey) according to VITA Shade has been forced, increasing an operator's discomfort. Further, although component-releasing dental restorative materials such as conventional antimicrobial fluorine-containing compositions, oral antibacterial agents, and halitosis removers have been developed, the conventional materials fail to have long-term release properties, so most components are initially released.

### Related Document

### Patent Document

1. Japanese Patent Application Laid-open No. 1987-086003

2. Japanese Patent Application Laid-open No. 1988-218703

### SUMMARY

In order to solve the above-described problems, the inventors of the present disclosure has found that when a filler is manufactured in a hierarchical structure of a core-shell structure using porous ceramics, it is possible to implement a color, by which the reflected light of the surrounding teeth color is not lost but transmitted to the outside so that the implemented color is not in contrast with the surrounding color when observed with the naked eye, and it is also possible that a color to be shown when particles of a certain size have a specific refractive index has the average L*, a*, and b* values of human teeth, thereby minimizing artificial color tones. In addition, when an oral active ingredient is supported in a porous structured filler, a multifunctional composite dental restorative resin may be developed by releasing the oral active ingredient for a long period of time, thus completing the present disclosure.

In one aspect, there is provided nanoparticles for a resin composition, the nanoparticles containing a core portion with an oral active ingredient and a porous ceramic shell portion coated on a part or all of a surface of the core portion.

In the present disclosure, the oral active ingredient may include a fluoride source, an anticariogenic agent, a calcium source, a phosphorus source, a remineralizing agent, enzyme, mouthwash, anesthetic, coagulant, acid neutralizer, a chemotherapeutic agent, an immune response modulator, thixotrope, polyol, anti-inflammatory, an antimicrobial agent, an antifungal agent xerostomia therapeutics desensitizer, or a combination thereof.

In the present disclosure, the porous ceramic may include silica, alumina, titania, yttria, zirconia, ceria, gallium oxide, latanium oxide, barium oxide, iron oxide, nickel oxide, cobalt oxide, copper oxide, zinc oxide, or a combination thereof.

In the present disclosure, an average particle diameter of the nanoparticles may be 200 to 400 nm.

In the present disclosure, the nanoparticles may be prepared by a method including: (a) preparing a dispersed phase solution by mixing a porous silica precursor and an organic solvent; (b) forming an emulsion by adding the dispersed phase solution to a continuous phase solution containing a surfactant; (c) heating the formed emulsion to remove the organic solvent; and (d) supporting the oral active ingredient by inducing gelation of the porous silica precursor.

In another aspect, there is provided a pigment-free and dye-free photocurable dental resin composition including a photocurable monomer component, a filler including nanoparticles for a resin composition according to the aforementioned aspect, and a polymerization initiator.

In the present disclosure, the photocurable monomer component may include a (meth) acrylic compound.

In the present disclosure, the photopolymerization initiator may include: benzoin methyl ether; benzoin ethyl ether; benzoin isopropyl ether; benzyl dimethyl ketal; benzyl ketals such as benzyl diethyl ketal; benzophenone; 4,4′-dimethylbenzophenone; 4-methacryloxybenzophenone; diacetyl; 2,3-pentadionebenzyl; camphorquinone; 9, 10-phenanthraquinone; 9, 10-anthraquinone; 2,4-diethoxythioxanthone; 2-chlorothioxanthone; methylthioxanthone; bis-(2,6-dichlorobenzoyl) phenyl phosphine oxide; bis-(2,6-dichlorobenzoyl)-2,5-dimethyl phenyl phosphine oxide; bis-(2,6-dichlorobenzoyl)-4-propyl phenyl phosphine oxide; bis-(2,6-dichlorobenzoyl)-1-naphthylphosphine oxide; bis-(2,4,6-trimethyl benzoyl)-phenylphosphine oxide; or combinations thereof.

In the present disclosure, the resin composition may further include a light stabilizer, and the light stabilizer may include Tinuvin P, avobenzone, or a combination thereof.

In the present disclosure, the resin composition may further include a polymerization inhibitor, an antioxidant, a viscosity modifier, a slush, a surface conditioner, a surfactant, an adhesion promoter, an antifoam, a slip agent, a diluent (solvent), a humectant, a stain repellant, a softener, a thickener, a polymer, or a combination thereof.

When applied to a dental restorative resin, the nanoparticles for a resin composition, the nanoparticles containing a core portion with an oral active ingredient and a porous ceramic shell portion coated on a part or all of a surface of the core portion, according to the present disclosure may improve aesthetics such as glossiness, maneuverability, color change before and after curing, color change over time after curing, and the like.

In addition, the nanoparticles for a resin composition according to the present disclosure enable the development of a long-term release dental restorative resin by supporting various oral active ingredients in a porous internal structure.

Meanwhile, the scope of the present disclosure is not limited by the effects as described above.

### BRIEF DESCRIPTION OF THE DRAWINGS

FIG. 1 shows an exemplary schematic diagram of the nanoparticles for resin composition according to the present disclosure.

FIG. 2 is a photograph showing tooth color harmony of a pigment-free and dye-free photocurable dental resin composition containing nanoparticles containing a core-shell porous structure according to the present disclosure.

### DESCRIPTION OF EXEMPLARY EMBODIMENTS

Hereinafter, a high transparency and long-release dental resin composition containing core-shell porous structured nanoparticles and a method for preparing the same according to embodiments of the present disclosure will be described in detail. However, these are only examples for illustrative purposes, and the scope of the present disclosure is not limited thereto. It is obvious to those skilled in the art that modifications to various embodiments are possible within the scope of the present disclosure.

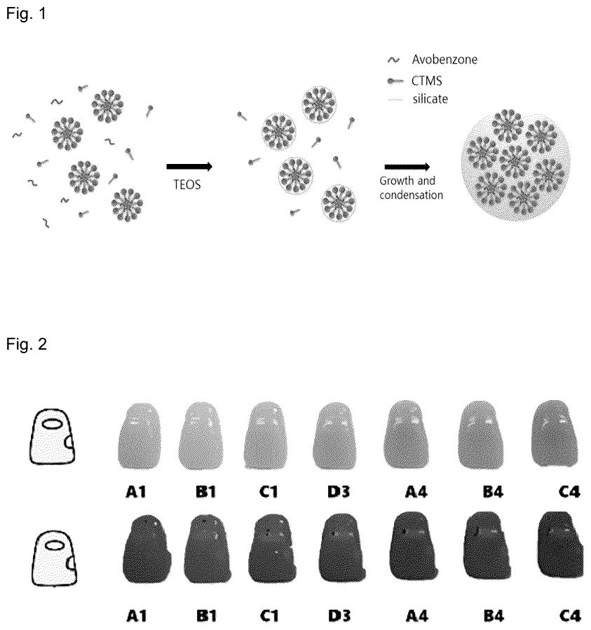

According to a first embodiment, the present disclosure provides nanoparticles for a resin composition, the nanoparticles containing a core portion with an oral active ingredient and a porous ceramic shell portion coated on a part or all of the surface of the core portion. An exemplary schematic diagram of the nanoparticles for resin composition according to the present disclosure is shown in FIG. 1.

In the nanoparticles for a resin composition according to the present disclosure, the oral active ingredient may include a fluoride source, an anticariogenic agent (e.g., xylitol), a calcium source, a phosphorus source, a remineralizing agent (e.g., calcium phosphate compound), enzyme, mouthwash, anesthetic, coagulant, acid neutralizer, a chemotherapeutic agent, an immune response modulator, thixotrope, polyol, anti-inflammatory, an antimicrobial agent (other than antimicrobial lipid components), an antifungal agent xerostomia therapeutics desensitizer, or a combination thereof.

In the nanoparticles for a resin composition according to the present disclosure, the porous ceramic may include silica, alumina, titania, yttria, zirconia, ceria, gallium oxide, latanium oxide, barium oxide, iron oxide, nickel oxide, cobalt oxide, copper oxide, zinc oxide, or a combination thereof.

In the nanoparticles for a resin composition according to the present disclosure, an average particle diameter of the nanoparticles may be 200 to 400 nm. When the diameter of the filler is out of the range, it may affect the texture of the surface of the resin composition, so the diameter may be optimized within the range.

In the nanoparticles for the resin composition according to the present disclosure, the nanoparticles may be prepared by a method including:
(a) preparing a dispersed phase solution by mixing a porous silica precursor and an organic solvent;
(b) forming an emulsion by adding the dispersed phase solution to a continuous phase solution containing a surfactant;
(c) heating the formed emulsion to remove the organic solvent; and
(d) supporting the oral active ingredient by inducing gelation of the porous silica precursor. The organic solvent may be ethyl acetate, dichloromethane, ethanol, acetone, methanol, or a combination thereof. The surfactant may be cetrimonium methosulfate (CTMS).

According to a second embodiment, the present disclosure provides a pigment-free and dye-free photocurable dental resin composition containing a photocurable monomer component, a filler and a polymerization initiator.

Here, the filler may be nanoparticles containing a core portion with an oral active ingredient and a porous ceramic shell portion coated on a part or all of a surface of the core portion.

In the photocurable dental resin composition according to the present disclosure, the photocurable monomer component may include a (meth) acrylic compound. For example, the (meth) acrylic compound may include: 2,2-bis(methacryloyloxyphenyl) propane; 2,2-bis[(3-methacryloyloxy-2-hydroxy propyl oxy)phenyl]propane; 2, 2-bis (4-methacryloyloxyphenyl) propane; 2,2-bis(4-methacryloyloxypolyethoxyphenyl) propane; 2, 2-bis (4-methacryloyloxydiethoxyphenyl) propane; 2,2-bis(4-methacryloyloxytetraethoxyphenyl) propane; 2,2-bis(4-methacryloyloxypentaethoxyphenyl) propane; 2,2-bis(4-methacryloyloxydipropoxyphenyl) propane; 2(4-methacryloyloxydiethoxyphenyl)-2(4-methacryloyloxytriethoxyphenyl) propane; 2(4-methacryloyloxydipropoxyphenyl)-2-(4-methacryloyloxytriethoxyphenyl) propane; 2,2-bis(4-methacryloyloxypropoxyphenyl) propane; 2,2-bis(4-methacryloyloxyisopropoxyphenyl) propane; 2-hydroxy ethyl methacrylate; 2-hydroxy propyl methacrylate; 3-chloro-2-hydroxy propyl methacrylate; ethylene glycol dimethacrylate; diethylene glycol dimethacrylate; triethylene glycol dimethacrylate; tetraethylene glycol dimethacrylate; neopentyl glycol dimethacrylate; 1,3-butanediol dimethacrylate; 1,4-butanediol dimethacrylate; 1,6-hexanediol dimethacrylate; 2-hydroxy ethyl methacrylate; 2-hydroxy propyl methacrylate; 3-chloro-2-hydroxy propyl methacrylate; hexamethylene diisocyanate; trimethylhexamethylene diisocyanate; diisocyanate methyl cyclohexane; isophorone diisocyanate; methylenebis(4-cyclohexyl isocyanate); trimethylolpropane trimethacrylate; trimethylol ethane trimethacrylate; pentaerythritol trimethacrylate; trimethylol methane trimethacrylate; pentaerythritol tetra methacrylate; pentaerythritol tetra acrylate; diisocyanate methyl benzene; diisocyanate methyl cyclohexane; isophorone diisocyanate; hexamethylene diisocyanate; trimethylhexamethylene diisocyanate; methylenebis(4-cyclohexyl isocyanate); 4,4-diphenyl methane diisocyanate; tolylene-2,4-diisocyanate; or a combination thereof. The content of the photocurable monomer component may be 1 to 98% by weight, based on a total weight of the resin composition.

In the photocurable dental resin composition according to the present disclosure, the oral active ingredient may include a fluoride source, an anticariogenic agent (e.g., xylitol), a calcium source, a phosphorus source, a remineralizing agent (e.g., calcium phosphate compound), enzyme, mouthwash, anesthetic, coagulant, acid neutralizer, a chemotherapeutic agent, an immune response modulator, thixotrope, polyol, anti-inflammatory, an antimicrobial agent (other than antimicrobial lipid components), an antifungal agent xerostomia therapeutics desensitizer, or a combination thereof.

In the photocurable dental resin composition according to the present disclosure, the porous ceramic may include silica, alumina, titania, yttria, zirconia, ceria, gallium oxide, latanium oxide, barium oxide, iron oxide, nickel oxide, cobalt oxide, copper oxide, zinc oxide, or a combination thereof.

In the photocurable dental resin composition according to the present disclosure, an average particle diameter of the nanoparticles may be 200 to 400 nm. When the diameter of the filler is out of the range, it may affect the texture of the surface of the resin composition, so the diameter may be optimized within the range. The content of the filler may be 0.01 to 5% by weight, based on a total weight of the resin composition.

In the photocurable dental resin composition according to the present disclosure, the photopolymerization initiator may include: benzoin methyl ether; benzoin ethyl ether; benzoin isopropyl ether; benzyl dimethyl ketal; benzyl ketals such as benzyl diethyl ketal; benzophenone; 4,4′-dimethylbenzophenone; 4-methacryloxybenzophenone; diacetyl; 2,3-pentadionebenzyl; camphorquinone; 9, 10-phenanthraquinone; 9, 10-anthraquinone; 2,4-diethoxythioxanthone; 2-chlorothioxanthone; methylthioxanthone; bis-(2,6-dichlorobenzoyl) phenyl phosphine oxide; bis-(2,6-dichlorobenzoyl)-2,5-dimethyl phenyl phosphine oxide; bis-(2,6-dichlorobenzoyl)-4-propyl phenyl phosphine oxide; bis-(2,6-dichlorobenzoyl)-1-naphthylphosphine oxide; bis-(2,4,6-trimethyl benzoyl)-phenylphosphine oxide; or combinations thereof. The content of the photopolymerization initiator may be 0.001 to 5% by weight, based on the total weight of the resin composition.

In the photocurable dental resin composition according to the present disclosure, the resin composition may further include a light stabilizer. The light stabilizer may be added as an ultraviolet absorber to increase weatherability of a cured product, and light stabilizer components known in the art may be used without limitation. For example, the light stabilizer may include Tinuvin P, Avobenzone, or a combination thereof. The content of the light stabilizer may be 0.001 to 5% by weight, based on a total weight of the resin composition.

In the photocurable dental resin composition according to the present disclosure, the resin composition may further include a polymerization inhibitor, an antioxidant, a viscosity modifier, a slush, a surface conditioner, a surfactant, an adhesion promoter, an antifoam, a slip agent, a diluent (solvent), a humectant, a stain repellant, a softener, a thickener, a polymer, or a combination thereof.

In the photocurable dental resin composition according to the present disclosure, the resin composition may be used as a dental adhesive material, a dental filling material, a resin for building dental supports, a pit and fissure sealant, a dental coating material, a dental cement, a desensitizer, or a dental fluoride varnish.

According to a third embodiment, the present disclosure provides a dental restorative material obtained by curing the photocurable dental resin composition. The restorative material refers to any type of restorative materials applied to dental applications.

In the dental restorative material according to the present disclosure, the restorative material may be prepared by a method including: providing the photocurable dental composition; applying the dental composition to a patient's teeth; and polymerizing the composition. According to one embodiment, the dental restorative material may have a Shore Hardness of 85 or more, and a depth of cure (DOC) of 1.5 mm or more measured according to ISO 4049 standard.

Hereinafter, the present disclosure will be described in more detail through examples and experimental examples. However, the following Examples and Experimental Examples are intended to illustrate the present disclosure, and the contents of the present disclosure are not limited to the following Examples and Experimental Examples.

### <EMBODIMENT>

Embodiment 1. Preparation of a resin composition containing core-shell porous nanoparticles

The porous silica nanoparticles according to the present disclosure is prepared by: preparing a disperse phase solution by mixing TEOS and ethyl acetate as a porous silica precursor; adding a solution in which CTMS is dispersed to the dispersed phase solution; and then firing the dispersed phase solution at 550° C. for 5 hours to remove CTMS.

Then, urethane-dimethylacrylate 4.23 g, 2,2bis[4-(methacryloxypolyethoxy]phenyl]propane, glycerol dimethacrylate of 11.38 g, butylated hydroxytoliene 0.01 g, titanium oxide 0.01 g, 0.08 ethyl-4-dimethyl aminobenzoate, and camphorquinone were mixed, and the porous silica nanoparticles prepared above were added and then mixed at 60° C. at a speed of about 35 rpm for 7 hours and 30 minutes. After every 1 hour and 30 minutes, a raw material adhered to a wall of a reactor was thoroughly scraped off, and this was repeated three times. Then, air bubbles were removed by adjusting temperature of the mixer to 25° C. and mixing at a rate of 10 rpm for 1 hour.

### <EXPERIMENTAL EXAMPLES>

Experimental Example 1. Evaluation of physical properties of a resin composition containing porous silica according to the present disclosure

Color change before and after curing, long-term color change after curing, compressive strength, refractive strength, and radiopacity of the porous silica nanoparticles prepared according to Example 1 were determined according to the ISO 4049, and then compared with the US, 3M, Filtek Z350 (Comparative Example 1), Japan, GC, Unifil (Comparative Example 2), Korea, SPIDENT, EsCom250 (Comparative Example 3) and Korea, Vericom, Denfil (Comparative Example 4).

TABLE 1

| | | | Comparative | Comparative | Comparative | Comparative |
|---|---|---|---|---|---|---|
| | | Example 1 | Example 1 | Example 2 | Example 3 | Example 4 |
| ΔE* for color | | 0.08 | 2.34 | 5.68 | 7.87 | 4.28 |
| before and | | | | | | |
| after curing | | | | | | |
| Long- | 30 days | 006 | 1.48 | 1.26 | 2.46 | 3.54 |
| term | 90 days | 0.18 | 1.65 | 2.06 | 2.54 | 3.68 |
| color | 180 days | 0.14 | 2.05 | 1.60 | 3.09 | 4.02 |
| change | | | | | | |
| after | | | | | | |
| curing | | | | | | |

| | | | | | |
|---|---|---|---|---|---|
| Compressive | 354 | 375 | 336 | 329 | 345 |
| strength (Mpa) | | | | | |
| Flexural | 158 | 162 | 147 | 139 | 128 |
| strength (Mpa) | | | | | |
| Radiopacity | 2.98 | 3.04 | 3.15 | 2.68 | 2.37 |
| (mm AI) | | | | | |

ΔE*={(ΔL*)2+(Δa*)2+(Δb*)2}1/2; ΔL*=L1*-L2*; Δa*=a1*-a2*; Δb*=b1*-b2*

(L1*: brightness index of cured product after storage, a1*, b1*: color quality index of cured product after storage, L2*: brightness index of cured product before storage, a2*, b2 *: color quality index of cured product before storage, ΔE*: Indicates the variation of color change)

As shown in Table 1, it was found that Example 1, which is a pigment-free and dye-free photocurable dental resin composition containing core-shell porous structured nanoparticles according to the present disclosure, stably maintains color without color change for a long time, compared to Comparative Examples 1 to 4. In addition, it is also found that Example 1 has properties equal to or superior to those of Comparative Examples 1 to 4 in terms of compressive strength, flexural strength and radiopacity.

Experimental Example 2. Evaluation of teeth color harmony of a resin composition containing porous silica according to the present disclosure

The pigment-free and dye-free photocurable dental resin composition containing core-shell porous structured nanoparticles according to the present disclosure was filled in the top and lateral sides of an artificial tooth, colored with representative seven tooth colors, and then observed with UV light.

As a result, as shown in FIG. 2, the pigment-free and dye-free photocurable dental resin composition containing core-shell porous structured nanoparticles according to the present disclosure was found to match with teeth of various colors.

The present disclosure is not limited to the specific preferred embodiments described above, and various modifications can be made by anyone having ordinary knowledge in the art to which the present disclosure belongs without departing from the gist of the present disclosure claimed in the claims. Of course, such changes are within the scope of the claims.

1. Nanoparticles for a resin composition, the nanoparticles containing a core portion with an oral active ingredient and a porous ceramic shell portion coated on a part or all of a surface of the core portion.

2. The nanoparticles of claim 1, wherein the oral active ingredient comprises a fluoride source, an anticariogenic agent, a calcium source, a phosphorus source, a remineralizing agent, enzyme, mouthwash, anesthetic, coagulant, acid neutralizer, a chemotherapeutic agent, an immune response modulator, thixotrope, polyol, anti-inflammatory, an antimicrobial agent, an antifungal agent xerostomia therapeutics desensitizer, or a combination thereof.

3. The nanoparticles of claim 1, wherein the porous ceramic comprises silica, alumina, titania, yttria, zirconia, ceria, gallium oxide, latanium oxide, barium oxide, iron oxide, nickel oxide, cobalt oxide, copper oxide, zinc oxide, or a combination thereof.

4. The nanoparticles of claim 1, wherein an average particle diameter of the nanoparticles for the resin composition is 200 to 400 nm.

5. The nanoparticles of claim 1, wherein the nanoparticles are prepared by a method comprising:
(a) preparing a dispersed phase solution by mixing a porous silica precursor and an organic solvent;
(b) forming an emulsion by adding the dispersed phase solution to a continuous phase solution containing a surfactant;
(c) heating the formed emulsion to remove the organic solvent; and
(d) supporting the oral active ingredient by inducing gelation of the porous silica precursor.

6. A pigment- and dye-free photocurable dental resin composition comprising a photocurable monomer component, a filler comprising the nanoparticles according to claim 1, and a polymerization initiator.

7. The dental resin composition of claim 6, wherein the photocurable monomer component comprises a (meth) acrylate compound.

8. The dental resin composition of claim 6, wherein the photopolymerization initiator comprises: benzoin methyl ether; benzoin ethyl ether;
benzoin isopropyl ether; benzyl dimethyl ketal; benzyl ketals such as benzyl diethyl ketal; benzophenone; 4,4′-dimethylbenzophenone; 4-methacryloxybenzophenone;
diacetyl; 2,3-pentadionebenzyl; camphorquinone; 9, 10-phenanthraquinone; 9, 10-anthraquinone; 2,4-diethoxythioxanthone; 2-chlorothioxanthone;
methylthioxanthone; bis-(2,6-dichlorobenzoyl) phenyl phosphine oxide; bis-(2,6-dichlorobenzoyl)-2,5-dimethyl phenyl phosphine oxide; bis-(2,6-dichlorobenzoyl)-4-propyl phenyl phosphine oxide; bis-(2,6-dichlorobenzoyl)-1-naphthylphosphine oxide; bis-(2,4,6-trimethyl benzoyl)-phenylphosphine oxide; or combinations thereof.

9. The dental resin composition of claim 6,
wherein the resin composition further comprises a light stabilizer, and
wherein the light stabilizer comprises Tinuvin P (Tinuvin P), Avobenzone, or a combination thereof.

10. The dental resin composition of claim 6, wherein the resin composition further comprises a polymerization inhibitor, an antioxidant, a viscosity modifier, a slush, a surface conditioner, a surfactant, an adhesion promoter, an antifoam, a slip agent, a diluent (solvent), a humectant, a stain repellant, a softener, a thickener, a polymer, or a combination thereof.
